(19) United States
(12) Patent Application Publication
(10) Pub. No.: US 2005/0037353 A1
(43) Pub. Date: Feb. 17, 2005

(54) Highly sensitive methods for protein detection in proteomics

(76) Inventors: David Y. Zhang, Jamaica, NY (US); Jizu Yi, Bayside, NY (US)

Correspondence Address: Steven B Pokotilow;Stroock & Stroock & Lavan, 180 Maiden Lane, New York, NY, 10038, US

(21) Appl. No.: 10/492,860

(22) PCT Filed: Oct. 15, 2002
(86) PCT No.: PCT/US02/33171

Related U.S. Application Data
(60) Provisional application No. 60/329,607, filed on Oct. 15, 2001.

(51) Int. Cl. C12Q 1/68; G01N 33/53; C12P 19/34

(52) U.S. Cl. 435/6; 435/91.2

(57) ABSTRACT

The present invention relates to methods for identification and quantification of proteins expressed within a cell. The methods of the invention involve the separation of proteins based on their physical properties such as, for example, net charge, molecular weight, or immunoreactivity, followed by detection of said proteins using a number of different techniques including (i) ramification-extension amplification method (RAM); (ii) hybridization signal amplification method (HSAM); and (iii) detection with nanodots. The methods of the invention will have a variety of different uses including, but not limited to, uses for screening, for diagnosis and prognosis of disease. The methods of the invention are especially useful for identification of proteins that are not easily identified due to the small size of the protein, low concentration of the protein, or failure to separate proteins due to similar physical properties.

### CROSS-REFERENCE TO RELATED APPLICATIONS

This application claims the benefit of U.S. Provisional Application Ser. No. 60/329,607, filed Oct. 15, 2001, titled HIGH SENSITIVE METHODS FOR PROTEIN DETECTION IN PROTEOMICS.

### BACKGROUND OF INVENTION

1. Field of the Invention

The present invention relates to methods for identification and quantification of proteins expressed within a cell. The methods of the invention involve the separation of proteins based on their physical properties such as, for example, net charge, molecular weight, or immunoreactivity, followed by detection of said proteins using a number of different techniques including (i) ramification-extension amplification method (RAM); (ii) hybridization signal amplification method (HSAM); and (iii) detection with nanodots. The methods of the invention will have a variety of different uses including, but not limited to, uses for screening, for diagnosis and prognosis of diseases. The methods of the invention are especially useful for identification of proteins that are not easily identified due to the small size of the protein, low abundance of the protein, or failure to separate proteins due to similar physical properties.

### BACKGROUND OF THE INVENTION

The simultaneous measurement of the abundance of many different protein species (i.e., proteomics) would be highly informative as to the specific status of dynamic cellular processes in normal development, in stages of disease, in response to drug treatment or gene therapy, or as a result of environmental exposure to other deliberate or inadvertent interventions. Gene expression profiling techniques permit the analysis of the expression levels of thousands of genes simultaneously both in health and disease. These systematic analyses will reveal potential and effective targets both for drug design for therapy and disease markers for diagnosis. Since gene-protein relationships have often been shown to be nonlinear, it is imperative to examine the protein profile of a cell in order to understand its underlying biological functions. Proteomics assays have significant advantages in identification, qualification, localization of target protein molecules, and studies of protein-protein interactions, all of which are essential in understanding the molecular mechanism of living cells.

In order to identify different protein expression profiles in heterogeneous tissue samples, one would need the capability to analyze the proteins expressed in a small number of cells. This capability is most relevant in the analysis of histological or cytological specimens that may harbor dysplastic or pre-malignant cells. Such cells, which may precede the development of cancer, need to be identified when present as small foci of 10 to 50 cells, before they have a chance to give rise to tumors. Unfortunately, the amount of protein obtained from 10 to 50 cells is insufficient for gel analysis, and is problematic even with the use of radioisotopes to label the protein. There is a need to detect and analyze the proteins in such small samples.

A number of different methods have been utilized for separation of proteins based on their inherent biological properties. Such properties include size, charge, conformation and/or immunoreactivity. Such techniques include conventional chromatography based on the size, charge or immunoaffinity of proteins, high performance liquid chromatography and electrophoretic separation of proteins.

The combination of two different electrophoresis methods has been widely utilized to separate proteins in complex mixtures such as tissues or body fluids. The first electrophoresis step generally separates proteins based on their net charges. The second electrophoresis step generally separates proteins based on their molecular weight. The use of two dimensional electrophoresis (2D gel electrophoresis) allows the simultaneous separation of up to several thousand individual proteins, providing an overall protein map of the protein mixture analyzed. The separated proteins can be visualized by means of staining with a variety of staining compounds including Coomassie blue or silver nitrate. Alternatively, the proteins containing isotopically labeled moieties such as with ³⁵S methionine, can be visualized by means of autoradiography.

However, there is a limitation of 2D gel technology due to the lack of a highly sensitive method to detect proteins that are present in very low abundance. In addition, current methods are not possible for quantitative comparison among a large number of resolved proteins whose abundance may span seven to eight orders of magnitude. Clearly, the sensitivity of silver nitrate and Coomassie blue staining is inadequate; new technology is highly demanded. The present invention provides methods designed to increase the sensitivity of protein detection in 2D gel electrophoresis.

Most significantly, 2D gel electrophoresis combined with Matrix Assisted Laser Desorption Ionization-Time of Flight Mass Spectrometry (MALDI-TOF MS), or tandem mass spectrometry (MS-MS) facilitates remarkably in identification of proteins, analysis of their modification, and protein-protein interaction. Mass spectrometry has become a powerful, rapid and sensitive tool for the analysis of proteins. All mass spectrometers are built of three components; (i) an ionization source such as MALDI; (ii) a mass analyzer; and (iii) an ion detector such as electron multipliers, photomultipliers or conversion dynode. Ions produced in the ion source are separated in the mass analyzer by their mass-to-charge ratio. MS data are recorded as “spectra” which display ion intensity versus the mass-to-charge value. Generally, the two types of MS data have been used for protein identification by correlation with sequence databases: (i) the accurate mass of peptides (within 5 ppm resolution) derived by specific enzymatic cleavage of the isolated protein; and (ii) spectra from individual peptides isolated after proteolysis of the target protein.

Also, as a complementary technology to DNA microarray for monitoring gene expression, proteomics holds great promise for the study of complex biological systems with applications in molecular medicine. Array-based methods for protein analysis affords a high-throughput format by which to study protein-protein, protein-DNA and protein-small molecule interactions, providing important functional information for newly identified genes derived from genome projects.

### SUMMARY OF THE INVENTION

The present invention relates to methods for identification and quantification of proteins expressed within a cell. The methods of the invention involve the separation of proteins based on their physical properties followed by detection of the proteins using a number of different techniques including (i) ramification-extension amplification method (RAM); (ii) hybridization signal amplification method (HSAM); and (iii) detection with nanodots. The methods of the invention will have a variety of different uses including but not limited to uses for screening, for diagnosis and prognosis of diseases. The methods of the invention are especially useful for identification of proteins that are not easily identified due to the small size of the protein, low abundance of the protein, or failure to detect the protein due to failure to separate proteins from one another.

### BRIEF DESCRIPTION OF THE DRAWINGS

FIG. 1 is a schematic diagram of amplification of a circularized probe by the ramification-extension amplification method.

FIG. 2 is a schematic diagram of hybridization signal amplification method.

FIG. 3 is a two dimensional gel stained with Coomassie blue.

### DETAILED DESCRIPTION OF THE INVENTION

The present invention relates to a highly sensitive method for identification and/or quantification of proteins expressed within a cell. The method of the invention involves the use of a variety of different methods to separate total proteins derived from cell extracts followed by detection of said proteins using a variety of different methods including (i) ramification-extension amplification method (RAM); (ii) hybridization signal amplification method (HSAM); and (iii) detection with nanodots. Specifically, the method for identifying and/or quantitating proteins expressed within a cell comprises the following steps:
(a) extracting proteins from a sample of cells;
(b) separating the extracted proteins;
(c) modifying the proteins; and
(d) detecting the proteins.

Methods for detecting proteins include (i) RAM; (ii) HSAM; and (iii) detection with nanodots.

A wide variety of protein mixtures can be prepared and separated into individual proteins using a variety of different methods. Proteins derived from cell extracts can be separated based on physical properties including size, charge, conformation and/or immunoreactivity. Such techniques include conventional chromatography, high performance liquid chromatography, electrophoretic separation of proteins, mass spectrometry and flow cytometry. Such separation methods are well known to those of skill in the art (See, for example, Ausebel et al., 1998, Current Protocols in Molecular Biology, John Wiley & Sons, Inc., V1.2, Chapter 10).

Prior to 2D gel electrophoresis, aliquots of cells are solubilized using any one of a variety of solubilization cocktails known to those of skill in the art. For example, tissue can be solubilized by addition of lysis buffer consisting of 8 M urea, 20 ml of Nonidet P-40 surfactant, 20 ml of ampholytes (pH 3.5-10), 20 ml of 2-mercaptoethanol, and 0.2 mM of phenylmethylsulfonyl fluoride (PMSF) per liter of distilled and deionized water.

In a specific embodiment of the invention, 2D gel electrophoresis may be used to separate the proteins. Methods of 2D electrophoresis are known to those skilled in the art. Electrophoresis in the first dimension generally separates proteins based on their net charges, while electrophoresis in the second dimension, referred to as sodium dodecyl sulfate-polyacrylamide gel electrophoresis (SDS-PAGE), separates proteins based on their differences in size (i.e., molecular weights).

One type of electrophoresis method that separates proteins based on their net charge is isoelectric focusing (IEF or carrier ampholyte based 2D gel electrophoresis). Carrier ampholyte based 2D gel electrophoresis can be done as previously described (Strahier et al, Journal of Clinical Investigtion, 85:200-207, 1990). IEF is generally used as the first phase of separation, or the first dimension, in 2D electrophoresis. Two proteins having different ratios of charged, or titrating, amino acids can be separated by virtue of their different net charges at some pH. Under the influence of an applied electric field, a more highly charged protein will move faster than a less highly charged protein of similar size and shape. If the proteins are made to move from a sample zone through a non-convecting medium (typically a gel such as polyacrylamide), an electrophoretic separation will result. For example, a more positively charged protein will move to a position with relatively higher pH established by the more net negative charges within the gel. When the total net number of positive and negative charges from both the protein and the gel are equal, the protein stops moving even under an applied electric field.

Because IEF is sensitive to charge modification, it is important to minimize protein alterations (e.g., proteolysis, deamidation of glutamine and asparagine, oxidation of cystine to cystic acid, carbamylation) that can result from improper sample preparation. Thus, once solubilized, samples should be stored frozen at −80° C. for short periods (<1 month) to limit significant protein modification.

Approximately 30 μl aliquots containing 70 μg of protein may be loaded onto individual gels, although the amount of protein to be loaded will vary depending on the type of detection method utilized. Prepared protein samples are loaded onto electrophoretic gels for IEF separation in the first dimension which separates proteins based on charge. In most cases aliquots are immediately applied onto IEF gels. First-dimension gels contain 50 ml of ampholytes per liter (pH 3.5-10). Generally, IEF is done at 1,200 V for 10 h and 1,500 V for the last 2 h. Twenty gels are generally run simultaneously.

A key requirement for an IEF procedure is the formation of an appropriate spatial pH gradient. This can be achieved either dynamically, by including a heterogeneous mixture of charged molecules (ampholytes) into an initially homogeneous separation medium, or statically, by incorporating a spatial gradient of titrating groups into the gel matrix through which the migration will occur. The former represents classical ampholyte-based IEF, and the latter the more recently developed immobilized pH gradient (IPG) IEF technique. The IPG approach has the advantage that the pH gradient is fixed in the gel, while the ampholyte-based approach is susceptible to positional drift as the ampholyte molecules move in the applied electric field. Current methodology combines the two approaches to provide a system where the pH gradient is spatially fixed but small amounts of ampholytes are present to decrease the adsorption of proteins onto the charged gel matrix of the IPG. A number of first dimension gel preparations may be utilized including tube gels for carrier ampholyte-based separations, or gel strips for immobilized gradient based separations.

Alternatively, IPG gels may be used (Hanash S. M., et al., 1991, Proc. Natl. Acad. Sci., USA 88:5709-5713). Samples are prepared using lysis buffer as discussed above. For first dimension separation an immobilized pH gradient covering the separation range of pH 4-10 is used. IPG gels are prepared using derivatives of acrylamide having carboxyl or tertiary amino groups with specific pK values. A linear pH gradient is prepared from a dense, acidic solution and a light, basic solution using a two-chamber microgradient former. The pH gradient is stabilized during polymerization of the Immobiline acrylamide-bisacrylamide matrix by a co-linear gradient of glycerol. Formulations of buffering Immobiline mixtures with titrating Immobiline for the pH limit solutions for narrow pH gradients (1 pH unit) or for broad pH gradients (>1 pH unit, up to 6 pH units) have been published (Gianazza et al, Electrophoresis 6:113 (1985) and LKB application Note 324 (1984)).

After first dimension separation, proteins are transferred onto the second dimension gel, following an equilibration procedure and separated using SDS-PAGE which separates proteins based on differences in their molecular weight.

Charged detergents such as SDS can bind strongly to protein molecules and “unfold” them into semi-rigid rods whose lengths are proportional to the length of the polypeptide chain, and hence approximately proportional to molecular weight. A protein complexed with such a detergent is itself highly charged (because of the charges of the bound detergent molecules), and this charge causes the protein-detergent complex to move in an applied electric field. Furthermore, the total charge also is approximately proportional to molecular weight (since the detergent's charge vastly exceeds the protein's own intrinsic charge), and hence the charge per unit length of a protein-SDS complex is essentially independent of molecular weight. This feature gives protein-SDS complexes essentially equal electrophoretic mobility in a non-restrictive medium. If the migration occurs in a sieving medium, such as a polyacrylamide gel, however, large (long) molecules will be retarded compared to small (short) molecules, and a separation based approximately on molecular weight will be achieved. This is the principle of SDS-PAGE electrophoresis as applied commonly to the analytical separation of proteins.

The second dimension separates proteins on the basis of molecular weight in an SDS gel. An 11.5 to 14% (2.6% cross-linking) acrylamide gradient provides effective separation of proteins having a mass of from 10,000 to 100,000 Da. Proteins outside this range may be less well resolved. Proteins with molecular weight less than 10,000 Da migrate close to the dye front and are less well resolved.

It is current practice to detect proteins in 2D gels either by staining the gels or by exposing the gels to a radiosensitive film or plate (in the case of radioactively labeled proteins).

Staining methods include dye-binding (e.g., Coomassie Brilliant Blue), silver stains (in which silver grains are formed in protein-containing zones) (Merril et al, Science, 211:1437-1438, 1961), negative stains in which, for example, SDS is precipitated by Zn ions in regions where protein is absent, or the proteins may be fluorescently labeled. In each case, images of separated protein spot patterns can be acquired by scanners, and this data reduced to provide positional and quantitative information on sample protein composition through the action of suitable computer software.

Alternatively, once the proteins have been separated from one another, they may be transferred to a matrix prior to detection. For example, when utilizing electrophoretic methods of separation, the proteins may be transferred to a membrane prior to detection. Such membranes include, but are not limited to, those used in Western blot analysis such as nitrocellulose. Alternatively, when using conventional chromatography methods or flow cytometry, the proteins may be detected directly as bound to the chromatographic material or as present in elutes.

Proteins immobilized on a membrane such as nitrocellulose are still capable of binding other molecules. Following separation, the proteins are transferred from the 2D gels onto membranes commonly used for Western blotting. The techniques of Western blotting and subsequent visualization of proteins are also well known in the art (Sambrook et al, “Molecular Cloning, A Laboratory Manual”, 2^{nd} Edition, Volume 3, 1989, Cold Spring Harbor). The standard procedures may be used, or the procedures may be modified as known in the art for identification of proteins of particular types, such as highly basic or acidic, or lipid soluble, etc. (See for example, Ausubel, et al., 1989, Current Protocols in Molecular Biology, Green Publishing Associates and Wiley Interscience, N.Y.).

In a modification of 2D gel electrophoresis, proteins can be separated using miniaturized 2D gel electrophoresis. Using this technique, both dimensions can be run on the same gel that has been cast to contain an IPG strip on one side of the gel and a SDS gel on the other side of the gel. In order to get appropriate separation of the proteins the electric current is switched after the first dimension electrophoretic step, before the second dimension separation. Such mini-2D gels can separate proteins more rapidly than conventional 2D gel electrophoresis and require a reduced amount of protein.

In a specific embodiment of the invention a RAM method, such as that described in U.S. Pat. No. 5,942,391, can be used to detect proteins bound to membranes. U.S. Pat. No. 5,942,391 is hereby incorporated by reference into this application in order to more fully describe the state of the art to which this invention pertains. Total proteins lysed from target cells are separated by 2D electrophoresis, and transferred to a membrane. Following transfer of the proteins onto the membrane, the proteins are modified by linkage to a ligand moiety such as biotin using a biotinylation reagent, i.e., biotinamidocaproic acid 3 sulfo-N-hydroxysuccinimide ester (Sigma-Life Science). The biotin-conjugated reagent is incubated with the membrane at a pH of between 6.5-8.5 resulting in covalent cross linking of the membrane bound proteins with a biotin molecule. Following linkage of the membrane bound proteins to a ligand moiety, single stranded nucleic acid molecules linked to a ligand binding moiety, such as streptavidin, are added followed by washing of the reaction mixture to remove any unbound reagent.

The term “ligand” as used herein refers to any component that has an affinity for another component termed here as “ligand binding moiety.” The binding of the ligand to the ligand binding moiety forms an affinity pair between the two components. For example, such affinity pairs include, inter alia, biotin with avidin/streptavidin, antigens or haptens with antibodies, heavy metal derivatives with thiogroups, various polynucleotides such as homopolynucleotides as poly dG with poly dC, poly dA with poly dT and poly dA with poly U. Any component pairs with strong affinity for each other can be used as the affinity pair, ligand-ligand binding moiety. Suitable affinity pairs are also found among ligands and conjugates used in immunological methods. The biotin/streptavidin affinity pair may be used in the subject invention.

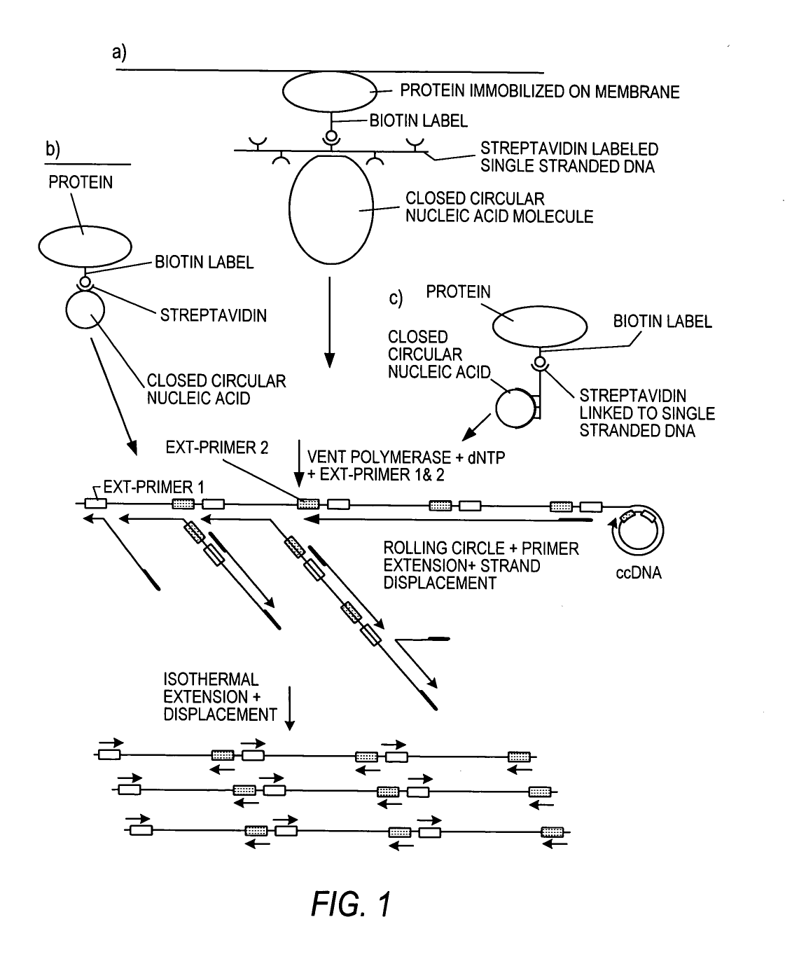

Applying RAM technology to the system, DNA polymerase and a closed circular nucleic acid molecule capable of binding to the streptavidin linked single stranded nucleic acid is added as a template. The streptavidin linked single stranded nucleic acid molecule acts as a primer for rolling circle replication (i.e., rolling circle amplification or RCA) of the closed circular nucleic acid molecule (FIG. 1). This results in linear amplification of the DNA sequences of the circular nucleic acid molecule. In another embodiment of the present invention additional primers complementary in sequence to the single stranded DNA product of the rolling circle replication are added which result in exponential amplification of the DNA sequences of the circular nucleic acid molecule. In a specific embodiment of the invention, labeled nucleotides may also be utilized and subsequently incorporated into the single stranded DNA with the primer extension so that the signal will be amplified. Such labels include, for example, fluorescent or radiolabeled-oligonucleotides. Detection may occur following either linear (RCA) or exponential (RAM) amplification of the circular nucleic acid molecule.

In another embodiment of the present invention, the proteins are separated by 2D gel electrophoresis and then modified directly while still in the gel. Then RAM or RCA, described herein above, or HSAM, described herein below, is performed.

The use of the RAM as an isothermal rolling circle amplification technique provides several unique advantages for proteomic studies. For example, sensitivity is increased due to replication/amplification of 10³ (from RCA)>10⁹ (from RAM) copies of DNA. Such increased sensitivity permits the detection of a single protein molecule. In addition, the method provides increased specificity due to lack of any detectable background signal. The increased sensitivity and specificity permit analysis of proteins from a single cell. In addition, the large magnitude of amplified nucleic acid permits quantitative analysis of the expressed proteins. Finally, radiolabeling is not required with RAM or RCA.

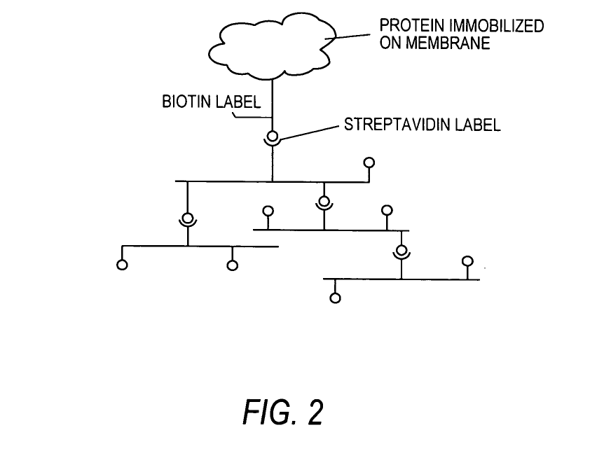

In another embodiment of the invention, HSAM, such as that described in U.S. Pat. No. 5,876,924, can be used to detect proteins bound to membranes. U.S. Pat. No. 5,876,924 is hereby incorporated by reference into this application in order to more fully describe the state of the art to which this invention pertains. Total proteins lysed from target cells are separated by 2D gel electrophoresis, and transferred to a membrane. Following transfer of the proteins onto the membrane, the proteins are modified by a biotinylation reagent, such as, for example, biotinamidocaproic acid 3 sulfo-N-hydroxysuccinimide ester (Sigma-Life Science). The biotin-conjugated reagent is incubated with the membrane at a pH of between 6.5-8.5 resulting in covalent cross linking of the membrane bound proteins with a biotin molecule. An oligonucleotide conjugated with multiple biotin molecules and a streptavidin molecule is then added to the membrane. The streptavidin molecule conjugated to the oligonucleotide will first interact with the protein conjugated biotins. The multiple biotin molecules conjugated to the oligonucleotide then provide additional sites for further streptavidin binding. The resulting branched-chain like reaction will lead to a formation of a large molecule for easy detection (FIG. 2).

In another embodiment of the present invention, a mixture of HSAM nanoparticles with different shapes and sizes can be added to the protein mixture following cell lysis. The HSAM nanoparticles can bind to the protein based on a specific, direct interaction. There are two forms of HSAM nanoparticles. In one form, the oligonucleotides that make up the HSAM nanoparticle contain specific sequences that form configurations that can interact with proteins specifically, such as DNA aptamers (Fredriksson, S et al., 2002 Nat Biotech 20: 473-477). In another form, the HSAM nanoparticle is packed into a matrix, such as agarose or acrylamide, to form a bead-like structure. The resulting beads have different surface shapes and charges that will interact with proteins similar to the nanodots, described herein below. No protein modification is necessary when using the HSAM nanoparticles.

In a further embodiment of the invention, nanodots, or nanoparticles (Elghanian et al., 1997, Science, 277:1078-81; Bruchez et al., 1998, Science 281:2013-16) can be used to detect proteins bound to membranes. After electrophoretic separation of the proteins as described above, detection can be carried out with nanodots, which are coated with active groups. The nanodots can be selected from, but are not limited to, the group consisting of quantum dots, metal dots, gold dots and polystyrene dots. In addition, mixed dots of different sizes and colors may be used to distinguish between different proteins. The active groups are designed to covalently link the dots to the target proteins bound to the membrane thereby immobilizing the nanodots. The quantity of immobilized dots is proportional to the amount of proteins in a target, therefore the present invention can be successfully employed for both protein detection and quantitative analysis. The cross-linking of proteins to nanodots provides a significant increase in sensitivity compared with current methods due to the fact that a single dot can be detected. Yet another advantage associated with the use of nanodots is that the proteins could immobilize nanodots selectively according to the match between the size of the dots and the surface of the protein rather than randomly. This selectivity provides a unique method to distinguish between different proteins that could not be separated by conventional 2D gel electrophoresis due to similarities in molecular weight and surface charges.

One limitation of current 2D gel electrophoresis technology arises when distinct proteins have similar charge and molecular weights, which makes it very difficult if not impossible to resolve them into distinct spots on the 2D gels. The result is one spot on the gel containing multiple proteins. One way to deconvolute the proteins is through the use of the specific binding characteristics of the nanodots. If nanodots with different protein binding characteristics and different colors are used then one could resolve the proteins into distinct populations. In addition, this detection of signal is dependent on a covalent interaction; therefore, the sensitivity of the detection can reach up to a single molecule.

In yet another embodiment of the present invention, proteins that bind to HSAM nanoparticles or nanodots can then be separated from each other and detected using flow cytometry, thereby foregoing the need for 2D gel electrophoresis. Because the dots contain different colors and have different shapes and sizes, when they pass through the detector each dot can be detected and recorded.

The following Experimental Details are set forth to aid in the understanding of the present invention, and are not intended, and should not be construed, to limit in any way the invention set forth in the claims which follow thereafter.

### EXAMPLE 1

### 2D Gel Electrophoresis

### Cell culture

PC-3 cells, derived from human prostate cancer, were purchased from the American Type Culture Collection (Manassas, Va.). The cells were grown in a 50:50 mixture of DMEM and F12 containing 1% antibiotic/antimycotic acids, and 10% Fetal Bovine Serum. The cells were incubated at 37° C. with a 5% CO₂ atmosphere.

PC-3 cells were treated with Scutellaria baicalensis, a Chinese herbal medicine, at a concentration of 0.6 mg/ml for 24 hours. The proteins were extracted and treated as described below.

Biotin-labeled Proteins

A stock labeling solution containing 2 mg/ml biotinamidocaproic acid 3 sulfo-N-hydroxysuccinimide ester (NHS-biotin) in 10% DMSO was prepared by dissolving 10 mg NHS-biotin (H1759FD; Sigma Chemical Co., St. Louis, Mo.) in 0.5-ml DMSO (Cryoserv, Research Industries Corp., Midvale, Utah), followed by addition of 4.5 ml PBS. This solution was sterilized by filtration through a 0.2 μm syringe filter (21062-25; Corning Glassware, Corning, N.Y.) made from DMSO-resistant materials.

PC-3 cultured cancer cells were harvested by treatment of trypsin-EDTA and then centrifugation. The cell pellets were washed by with 1×PBS for three times. The total protein from cell lysates was adjusted with PBS to 1.0 mg/ml. The appropriate volume of NHS-biotin was added to give a final concentration of NHS-biotin to 1 μg/ml. The sample was incubated at room temperature for 30 min., followed by dialysis with 20 mM Tris-HCl buffer with 5 M Urea for 12 hours to remove excess unreacted NHS-biotin. The protein was then concentrated to 1-2 mg/ml by centrifugation with Ultrafree-CL (UFC4LCC 25; Millipore, Bedford, Mass.), and the sample buffer was changed to 20 mM Tris/HCl buffer containing 7M urea, 2% CHAPS, 50 mm DTT. At this point the sample was ready for 2D gel analysis.

2D Gel Electrophoresis

0.5 ml protein suspended in 20 mM Tris/HCl buffer containing 7M urea, 2% CHAPS, 50 mm DTT was added to a channel in a re-hydration/equilibration tray (Bio-Rad Laboratories). A Readystrip IPG strip gel was placed side down onto the sample. 2 to 3 ml of mineral oil were added to cover the strip to prevent evaporation during the re-hydration process. The tray was placed on a leveled bench overnight to allow the protein samples to be absorbed into the IPG strips, which has a pH gradient. Then the IPG strip was transferred into a focusing tray. The first dimension gel electrophoresis based on pH gradient on the strip was carried out using the PROTEAN IEF apparatus according to the protocols provided by the manufacturer (BIORAD Laboratories) with a maximum current of 50 μA/strip and 10,000 V for 12h. Hereafter, the IPG strips were washed with equilibration buffer I (6M urea, 0.375 M Tris pH 8.8, 2% SDS, 20% glycerol, 2% DTT) for 10 min and followed by washing with equilibration buffer II (6M urea, 0.375 M Tris pH 8.8, 2% SDS, 20% glycerol, 2.5% iodoacetamide) for 10 min.

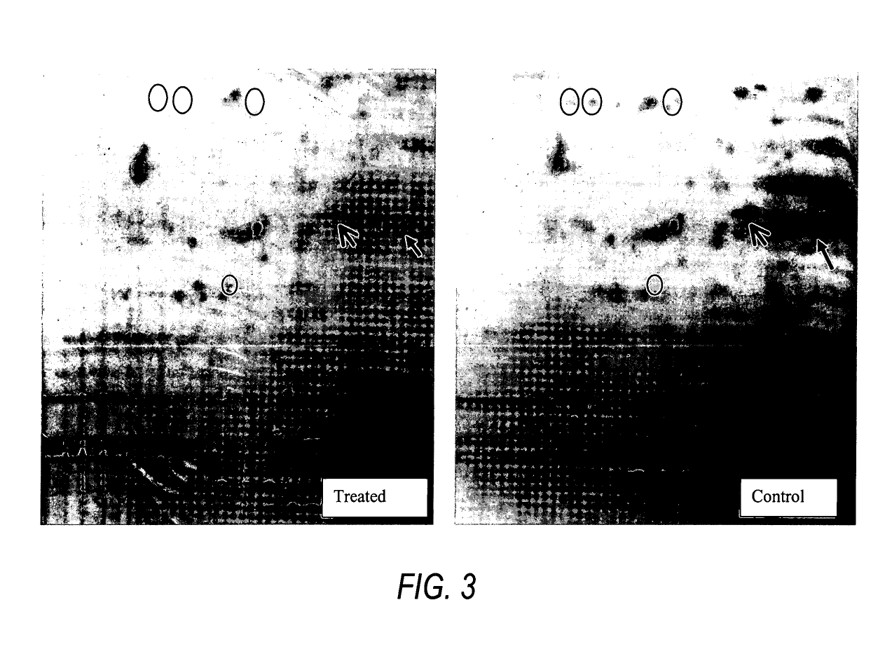

The equilibrated gel was then ready for the second dimension gel electrophoresis (SDS-PAGE) based on the sizes of the proteins. For SDS-PAGE, the strip gel was placed side-up on the top of an 8-14% polyacrylamide gel, and then covered with 0.5-1% agarose. After the agarose was polymerized, the buffer for the SDS-PAGE was added, and the electrophoresis was then carried out at 200 V for 5-5.5 h. The gels (treated and control) were stained with Coomassie blue (FIG. 3). The result of the 2D gel electrophoresis was the separation of the proteins from the mixture. The preliminary results demonstrate that there are significant changes in the protein expression levels in the treated cells when compared to the control or untreated cells, including decreases (indicated by the circles near the top of the gel), increases (indicated by the large arrow), new protein synthesis (indicated by the circle in the middle of the gel) as well as shut down of protein synthesis (indicated by the small arrow).

While there have been described what are believed to be the preferred embodiments of the invention, it will be recognized by those skilled in the art that other and further changes may be made thereto without departing form the spirit of the invention and it is intended to claim all such changes and modifications as fall within the true scope of the invention.

1. A method for detection of a protein in a sample comprising:
(a) extracting the protein from the sample;
(b) separating the protein from other proteins;
(c) modifying the protein with a ligand;
(d) contacting the protein with a nucleic acid comprising a ligand binding moiety and a region complementary in sequence to a circular oligonucleotide probe;
(e) adding the circular oligonucleotide probe, comprising a region complementary in sequence to the nucleic acid;
(f) adding a DNA polymerase; and
(g) amplifying the circular probe, wherein detection of the amplification of the circular probe indicates the presence of the protein in the sample.

2. The method of claim 1, wherein the protein is transferred to a membrane following step (b).

3. The method of claim 1, wherein the circular probe is amplified using an amplification method selected from the group consisting of polymerase chain reaction, strand displacement amplification, transcription mediated amplification, rolling circle amplification, RAM and primer extension.

4. The method of claim 3, wherein the amplification method is RAM.

5. The method of claim 3, wherein the amplification method is rolling circle amplification.

6. The method of claim 1, wherein the circular probe is amplified in the presence of labeled nucleotides.

7. The method of claim 18, wherein the nucleic acid is detected via HSAM.

8. The method of claim 1, wherein the ligand is selected from the group consisting of biotin, digoxigenin, antigens, haptens, antibodies, heavy metal derivatives, and polynucleotides.

9. The method of claim 1, wherein the ligand binding moiety is selected from the group consisting of strepavidin, avidin, anti-digoxigenin antibodies, antibodies, antigens, thio groups and polynucleotides.

10. A method for detection of a protein in a sample comprising:
(a) extracting the protein from the sample;
(b) separating the protein from other proteins;
(c) contacting the protein with a nanodot comprising active groups; and
(d) detecting the nanodots, wherein detection of the nanodots indicates the presence of the protein in a sample.

11. The method of claim 10, wherein the nanodot is selected from the group consisting of quantum dot, metal dot, gold dot and polystyrene dot.

12. The method of claim 11, wherein the nanodot is a quantum dot.

13. A method for detection of a protein in a sample comprising:
(a) extracting the protein from the sample;
(b) contacting the protein with a HSAM nanoparticle, thereby forming a protein/HSAM nanoparticle complex; and
(c) detecting the complex, wherein detection of the complex indicates the presence of the protein in the sample.

14. The method of claim 13, wherein the HSAM nanoparticle comprises an oligonucleotide that can bind to proteins.

15. The method of claim 13, wherein the HSAM nanoparticle comprises a matrix thereby forming a configuration that can form a complex with a protein.

16. The method of claim 13, wherein the complex is separated using flow cytometry.

17. The method of claim 13, wherein the complex is detected using flow cytometry.

18. A method for detection of a protein in a sample comprising:
(a) extracting the protein from the sample;
(b) separating the protein from other proteins;
(c) modifying the protein with a ligand; and
(d) contacting the protein with a nucleic acid comprising a ligand binding moiety, wherein detection of the nucleic acid indicates the presence of the protein in the sample.

19. The method of claim 18, wherein the protein is transferred to a membrane following step (b).

20. The method of claim 18, wherein the ligand is selected from the group consisting of biotin, digoxigenin, antigens, haptens, antibodies, heavy metal derivatives, and polynucleotides.

21. The method of claim 18, wherein the ligand binding moiety is selected from the group consisting of strepavidin, avidin, anti-digoxigenin antibodies, antibodies, antigens, thio groups and polynucleotides.
